(19) United States
(12) Patent Application Publication
(10) Pub. No.: US 2025/0391525 A1
(43) Pub. Date: Dec. 25, 2025

(54) AI-BASED CALCULATION OF A CASE COMPLEXITY INDEX

(71) Applicant: Siemens Healthineers AG, Forchheim (DE)

(72) Inventors: Ingo Schmuecking, Yardley, PA (US); Puneet Sharma, Princeton Junction, NJ (US); Arijit Bhattacharyya, Erlangen (DE)

(21) Appl. No.: 19/239,078

(22) Filed: Jun. 16, 2025

(30) Foreign Application Priority Data
Jun. 24, 2024 (EP) 24183908.3

(51) Int. Cl. G16H 10/60; G06V 10/774; G06V 10/82

(52) U.S. Cl. CPC G16H 10/60; G06V 10/774; G06V 10/82; G06V 2201/03

(57) ABSTRACT

Systems and method for an AI-based calculation of a case complexity index, CCI. For training a neural network, NN, the method includes receiving training data comprising a medical image of a set of medical images and for example a related report for the medical image and a CCI for the medical image. The method may further include training the NN for providing a trained NN, that is configured for determining the CCI for a medical image by adjusting weights and biases of the NN such that a loss function is minimized.

### CROSS REFERENCE TO RELATED APPLICATIONS

This application claims the benefit of EP 24183908.3 filed on Jun. 24, 2024, which is hereby incorporated by reference in its entirety.

### FIELD

Embodiments relate to the field of medical image processing and related tasks and to the determination of a case complexity index for use in processing of medical images or related tasks based on the medical images in the field of medical technology.

### BACKGROUND

Imaging service providers, e.g. radiology departments, teleradiology companies, radiology as a service (RaaS) providers, need to optimize efficiency and throughput of image reading and reporting while achieving best possible quality of care (clinical-economical optimization). This requires assignment of cases to the right readers and overall reading worklist prioritization. It also requires precise understanding of the effort (time and difficulty) required to accomplish reading or other image-related tasks.

This represents a complex problem for imaging service providers with high volumes (100k 1m+ procedures per year), broad procedure mix, and a large pool of radiologists with varying levels of experience and skills.

Imaging exams, such as chest imaging, have a small set of common findings and a large set of uncommon and rare findings known as “long-tail” distribution. There are about 260 imaging findings in chest imaging caused by 2958 different disorders (see e.g., Kahn CE. The Long Tail. Radiology Artificial Intelligence. Posted Apr. 18, 2019, https://pubs.rsna.org/page/ai/blog/2019/4/the_long_tail). An imaging service provider with a pool of general and chest radiologists may want to assign “easier” chest radiographs (e.g. with common findings) to general radiologists and free up chest radiologists to read chest CT exams while also handling “more difficult” chest radiographs (e.g. from the long-tail distribution) to make sure that they are accurately interpreted in case of rare disorders.

Therefore, there is a need in the art to provide an objective basis to control image-based subsequent tasks, for example a reading task.

The imaging IT environment used by radiology providers may include a reading worklist. The reading worklist enables physicians to see unread imaging studies including patient information, imaging modality, procedure type, diagnosis/reason for the exam, referring physician, and flags indicating urgent exams. The worklist may also display how long the study has been unread and when it needs to be read as per turnaround time agreements. Throughput and efficiency statistics may be generated including number of cases read, reading times etc.

With workflow orchestration software, it is possible to efficiently implement a wider set of processing rules to determine the assignment of cases to reading physicians. However, these approaches may be made without analysis of the actual imaging data which need to be read.

In clinical practice, physicians may pick cases from the worklist based on their preferences, e.g. less experienced physicians may choose “easier” cases based on a few simple criteria, such as age, care setting, referrer. Also, it may occur that physicians pick procedures with higher reimbursement over procedures with low reimbursement, that may lead to a non-optimal assignment of the medical images to a medical practitioner and/or to a computing device with appropriate computing resources.

In state of the art, AI tools such as computer-aided triage and notification (CADt) software may be used to analyze specific imaging studies to mark cases with urgent findings. These studies may be prioritized over other studies not marked by the software. However, this does not reduce the overall effort involved in reading the entire worklist.

AI tools may also be used after image reading and reporting is completed to support quality assurance tasks. However, this is a retrospective approach after the radiology service is provided.

None of these approaches discloses a method for providing an objective metric, based on measurement data, relating to the medical images for controlling subsequent image-based processing tasks, for example reading tasks, assignment to computing devices etc.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide an improvement for the image-based processing, for example with respect to time and/or quality.

In the following, embodiments are described with respect to the methods (training and inference) first. Features, advantages, or alternative embodiments mentioned with respect to the methods may be assigned to the other claimed objects (e.g. the computer program or a device, for example, the CCI-calculator device, or system or a computer program product) and vice versa. In other words, the system, apparatus, or device (claims) may be improved with features described or claimed in the context of the method and vice versa. In this case, the functional features of the method are embodied by structural units of the apparatus or device or system and vice versa, respectively. The method may refer to a software implementation and the device may refer to a hardware implementation. In computer science a software implementation and a corresponding hardware implementation (e.g. as an embedded system) are equivalent. Thus, for example, a method step for “receiving input data” may be performed with an input interface (and as known for a person skilled in the art with respective instructions to read data). For the sake of avoiding redundancy, although the device may also be used in the alternative embodiments described with reference to the method, these embodiments are not explicitly described again for the device. In principle, the respective device or apparatus claim is configured to carry out the method.

According to a first aspect, embodiments provide a computer-implemented method for training a neural network, NN, for determining a case complexity index, CCI, comprising the following method steps: receiving training data, comprising: a medical image of a set of medical images and for example a report for the medical image; a CCI for the medical image; training the NN for providing a trained NN, that is configured for determining the CCI for a medical image and for example a related report by adjusting weights and biases of the NN such that a loss function is minimized.

The CCI may be calculated based on a contradicting score. The contradicting score in turn may be calculated on an evaluation of a degree of complementary and/or contradicting and/or inconsistent information between the medical image and the report. A rule stored in a rule database may be used to apply a function for automatically calculating the contradicting score. For example, the higher the degree of complementarity and/or contradiction and/or inconsistency between the medical image and the report, the higher the CCI.

In an embodiment the CCI may be determined by using the formula: CCI=k/cs, where cs represents the contradicting score and k represents a constant factor for controlling the degree of proportionality.

The CCI may be calculated on the basis of time for reading the medical images (reading time) and/or a difficulty level. The difficulty level may relate to the difficulty for interpreting the medical images and/or may be calculated by a difficulty estimator.

The difficulty estimator may be or include a machine learning method. The difficulty estimator may be trained on data with ground truth provided by clinical experts based several attributes. The attributes may be selected from the list consisting of: reason for exam, number of prior exams as an indicator of complexity, number of images that need to be reviewed, number of findings: studies no findings, single findings or multiple findings, tasks that need to be performed: e.g. lung nodules measurements with a diameter on axial slices requires less effort than volumetric lung nodule measurement, and quality assurance (QA) data: As part of quality assurance, radiology providers select a sample of studies and have them read by a different physician who indicates agreement with the original report, minor disagreement or major disagreement. The QA result may be used as a parameter to estimate case difficulty.

The CCI is an index for indicating a complexity for a downstream task to be executed on the medical image, for example a reading task. The reading task may include an annotation task.

The CCI may be represented as a digit or figure. The CCI may be normalized for example as a figure in a value range, e.g. in an interval between 0 and 100. The CCI may serve as metric to assess complexity of image processing. The CCI may e.g., represent an estimated execution time of an annotation task (e.g. a reading time). Alternatively or in addition the CCI may represent a difficulty level of the annotation task (e.g., indicating a required skill or education level of the annotator, also denoted as clinician skill level).

In an embodiment it is possible to store the image data differently depending on the calculated CCI. For example, the complex cases are stored additionally on a special computer and the less complex ones only in the cloud.

In addition or alternatively, subsequent processing of the medical images may be controlled differently based on the calculated CCI. For example, the assignment of the medical images to medical professionals and/or the processing tools to be used for processing the medical images may be controlled differently. For example, the scenarios may be as follows: cases may be assigned to different clinicians in the reading worklist depending on the CCI; reading worklists across clinicians may be balanced depending on the CCI to make sure that all clinicians manage comparable case difficulty; cases may be processed with different AI tools depending on the CCI, e.g. cases with low difficulty may not need to be processed by a task-specific AI tool and cases with high complexity may get processed by a task-specific AI tool; depending on the CCI, studies may be selected for a pre-annotation service which provides preliminary report and annotations for review by the radiologist who will finalize the report; depending on the CCI, studies may be assigned to different pre-annotation service providers and/or depending on the CCI, some images may be routed to an AI-only read, while other may have a human expert in the loop. For example, a threshold may be defined for the different routings.

The CCI may serve to calculate a configuration setting and/or a prioritization for a downstream task, such as an annotation task for a set of images.

The training data include a medical image.

The training data may include a medical image and a related report in addition to an associated CCI. In this embodiment, the training dataset includes a pair of “image and related report.” The pair is a multi-modal dataset. The pair includes two different elements, that are acquired from different data sources (e.g. a reporting computer and an image acquisition device or a storage means for images). The pair is a digital dataset that may be stored in a tuple form or as a two-element matrix, including at least a medical image and a report related to this image, the elements of the pair may be provided in different formats. The image is provided in an image format for medical images, for example in a DICOM format and/or the report may be provided in a text format. Radiology reports may be text based without specific formats. Radiology reports may be created using free text and/or structured reporting which provides a more controlled structure or vocabulary. Radiology reports may include structured having sections, such as: clinical history (reason for the exam), prior comparison: no or yes (exam and date), findings, and/or impression.

Alternatively or in addition, the training method, and/or the method for calculating the CCI may use a difficulty estimator. The difficulty estimator may be configured to estimate the difficulty or complexity to further process the image. The difficulty estimator may be related to an image and/or to a downstream task and/or may serve to assess the difficulty and/or complexity of subsequent tasks, such as reading, annotating, reporting, and/or other software-supported tasks to be executed on the image. The difficulty estimator may use information from different sections (for example in the image, meta data, DICOM header data, etc.) to estimate the difficulty/complexity. The difficulty estimator may be implemented algorithmically or may be implemented as machine learning algorithm.

The NN that is trained for determining the CCI may have a feedforward architecture, for example a perceptron architecture with an input layer, a hidden layer, and an output layer. However, it is also possible to use different architectures, such as recurrent neural networks (RNNs) and convolutional neural networks (CNNs), that have different structures and are configured for particular types of (input) data and (e.g., annotation) tasks.

The NN may be a Vision Language Model that may take as input the image and text report simultaneously, and use dedicated image-only and text-only encoders with contrastive learning. Alternatively, the NN may also have joint embeddings of image and text.

The NN may be configured for a regression task, for example for determination of the CCI.

Each connection between nodes in adjacent layers may be associated with a weight, that determines the strength of the connection. During the training process, the weights are adjusted based on the error between the predicted output and the actual output. For this purpose, known techniques may be used for example backpropagation and gradient descent.

The loss function (or objective function) is used to measure the difference between the predicted values of a model (CCI for the pair) and the actual values in the training data. For example, a Mean Squared Error (MSE) for quadratic penalization of the model failures, a Mean Absolute Error (MAE) for penalizing errors linearly, that makes it less sensitive to outliers compared to MSE may be used. But also other loss functions, for example the Huber Loss, cross-entropy loss/log loss may be applied.

The term image data refers to medical images, for example provided in a DICOM format. The images may be acquired by different imaging procedures, for example CT, x ray, tomosynthesis, MRI, PET, SPECT, ultrasound, and others. The images may represent an anatomical structure of a patient, for example chest, chest CT, and/or chest radiographs. The image may alternatively refer to other anatomical structures, for example the heart, liver, intestine etc.

Alternatively or cumulatively the training data may further include at least one of the following data elements: clinical data for the respective patient, the set of medical images refers to; operational data and/or guideline data.

Clinical data may refer to data defining the medical task and/or medical setting. Clinical data may include a medical question (e.g., exclusion of a pathology, diagnoses of a particular disease etc.), known diagnoses, available documentation for the patient (to whom the image refers to), and/or prior images (e.g. prior studies and/or prior reports), and/or available medical data, such as lab data. Lab data refer to data of a medical laboratory analysis, including biochemical, image-based, tumor-marker-based, hormonal, genetical, and/or microbiological analysis, of e.g. blood or other body fluids. Alternatively or in addition, clinical data may include demographic data, co-morbidities, and/or therapy related data, for example already executed therapies (surgical intervention, chemo therapy etc.).

Operational data may refer to data defining the procedure which is to be executed on the medical image data. Operational data may include time data, indicating how much time does it take to execute the procedure. In addition or alternatively, operational data may include storage data, indicating from which source and/or storage the image data are provided, which type of downstream task is to be executed or expected to be executed on the image data, for example a reporting task, an annotation task etc.

Guideline data may refer to data, provided in medical guidelines, such as guidelines of the WHO (World Health Organization), AHA (American Heart Association) and/or guidelines of the SCCM (Society of Critical Care Medicine). The SCCM produces guidelines for the management of critically ill patients in intensive care units (ICUs). Guideline data may be based on the provisions of the Radiological Society of North America (RSNA). For example, an example for guidelines for diagnostic imaging may e.g., be the Guidelines for Management of Incidental Pulmonary Nodules Detected on CT Images: From the Fleischner Society 2017. https://pubmed.ncbi.nlm.nih.gov/28240562/.

Alternatively or in addition, the guideline data may be a local/organization-level SOP (standard operating procedure).

Guidelines data may include glossary data, e.g., SNOMED for calibration or harmonization of terminology.

Guidelines data may be actualized according to a predefined update setting. This has the technical advantage that the NN is forced to be trained on actualized guidelines data.

A calibration algorithm may have the functionality to map different terms having the same meaning (for example disease, illness) to a concurring term. The calibration algorithm may use guidelines or guideline data.

Alternatively or in addition, the CCI may be used for configuring or controlling an image annotation task, for example with respect to estimated reading time, and/or clinician skill level.

Alternatively or in addition, the set of medical images may include current images and/or prior images of the same or of a related procedure, for example of the same patient.

In an embodiment, the images in the set of medical images refer to the same patient.

Alternatively, it is possible that the images in the set of medical images refer to other patients with comparable settings. The comparable settings may include the type of procedure to be executed on the medical image and/or the type of annotation task to be executed on the image. Alternatively or in addition, comparable settings may also include clinical settings, e.g. outpatient, inpatient, intensive care unit or general hospital vs specialty hospital.

Alternatively or in addition, the method may include: using a calibration module, that is configured for calibrating the received training data, for example with respect to different images and/or reports.

It is to be noted that the calibration module may host different instances of a calibration algorithm that is configured for executing the step of calibrating. One instance may refer to calibrating the training data, as mentioned above and another instance may refer to calibrating input data in inference phase. This may be used as preprocessing for the input data in order to map the terms of the textual input data into terms which were used during training. In general, “calibration” refers to both instances and may relate to a calibration algorithm.

Alternatively or in addition, calibration may relate to calibrate different types of reports, including different level of details, different terminology used, different wordings of the authors of the report and/or different structuring, for example a structured report versus a free text report.

Calibration may alternatively of in addition relate to a location-based calibration, e.g., a calibration for different medical institutions at different locations and/or sites. Thus, site-specific data may be considered for the determination of the CCI. For example in that e.g., an academic hospital or a specialty hospital for e.g. cardio-vascular diseases generally requires a more detailed analysis than a primary care center providing care for an outpatient population. The type of site (clinic) may be identified by its location or position. Therefore, the location may be considered for CCI determination.

Calibration may alternatively or in addition relate to adapting or calibrating texts, which have been generated by using different linguistic concepts. For this purpose, an ontology may be used to transfer different terms with the same or similar meaning into a common term. An example for an ontology is the Fleischer Society: Glossary of Terms for Thoracic Imaging https://pubs.rsna.org/doi/10.1148/radiol.232558. The term “Nodule” may serve as an example.

The calibration task may refer to a computer-implemented task for calibrating data of different patients for a similar medical question or a similar annotation task, data of the same patients, but from different studies and/or procedures and/or from different image acquisition times and/or data of reports from different authors (language transfer).

Alternatively or in addition calibration may relate to: population with high vs. low percentage of normal cases, population with high vs low percentage of cases requiring comparison of current and prior studies, differences in image acquisition and reconstruction, low-dose vs standard dose chest CT (standard dose having better image quality), chest CT with 1 mm slice thickness vs 3 mm slice thickness (3 mm has fewer images), different procedure mix, and/or different reporting standards, e.g. reports where all findings (normal and abnormal) are described in detail vs reports which only mention abnormal findings.

Alternatively or in addition, the NN may include at least one of a vision language model, a large language model, and/or an image processing model.

In another aspect, embodiments provide a method for calculating a case complexity index, CCI, for an input dataset, for example including at least one medical image and for example a related report by using a trained neural network, NN, that has been trained with a method as described above. This aspect refers to the inference phase, during which the trained NN is applied for unseen cases.

The input dataset may be preprocessed. For example, the input dataset may be calibrated by a calibration module or a calibration algorithm adapted for use in the inference phase, for example for harmonizing the input dataset.

Input data include image data, that are acquired on a medical scanner (CT, MRI, US, PET etc.). The raw data may be subject to post processing to provide the respective images. The images may be stored in a local or cloud-based memory. The images are subject to further processing by executing downstream tasks. Downstream tasks may be implemented by software and/or hardware. A downstream task may include an annotation task.

Before executing the downstream task on a computing device, the method for calculating the CCI may be used. For example, the CCI may be used to configure the computing entity for the specific task with estimated resources, that are estimated on the basis of the CCI. The CCI may be used to control the computing entity and/or the data transfer to the same on the basis of the CCI (the CCI indicating bandwidth and data transmission requirements). The CCI may be used to control computing resources of the computing entity, which is selected to execute the downstream task. The CCI may alternatively or in addition be used to select a computing entity which has the appropriate resources according to the CCI (e.g., software licenses and/or processing power and/or other resources required for complex cases, etc.). The CCI may be used to configure a device and/or software, implemented on that device for a downstream task.

For example, the CCI may be used to trigger other devices to perform additional image reconstructions from raw imaging data, e.g. trigger image reconstruction of a chest CT with thinner slice thickness based on the case complexity.

The CCI may be used to select among a multitude of AI devices which AI device(s) should be used for a given study, e.g. a case with high complexity due to extensive infectious disease in the lungs may not benefit from an algorithm intended to detect pulmonary nodules.

The CCI may be used to select which AI operating point to use for a given study, e.g. high-sensitivity or high-specificity mode. For example, an imaging study with low complexity due to lungs without presence of disease patterns may trigger to run an algorithm for detection of pulmonary nodules at a higher sensitivity operating point.

The CCI may also be used to sort the reading worklist in which a clinician should read the images, e.g. starting with the most complex cases at the beginning of the reading session.

Cases with very high complexity may be added to additional reading worklists for reading by two clinicians.

The CCI may be used to select cases which may be used as input for AI algorithm training and store them in a designated location for this purpose that may be shared with the AI developer after de-identification.

The CCI may be used to select cases for quality assurance processes and store them in a designated location for this purpose.

In addition or alternatively, the determination of the CCI may consider a typical long tail distribution of findings, see also Kahn et al. Thus, it is be taken into account that there is a small set of common findings and a large set of uncommon or rare findings. The CCI may be tailored (configured) to assign higher complexity to rare findings compared to common finding, for example with respect to the long tail distribution.

The preprocessing may in addition or alternatively include an uncertainty module, which is configured for calibrating uncertainties in the report. For example, slightly different formulations in the report may still refer to the same content or same formulations in the report may refer to different imaging findings. Also, reports are expected have inherent inter-reader variability due to perception and interpretation challenges. This is taken into account when training and/or applying the NN in the inference phase.

The determined CCI may be used for configuring a downstream task, to be executed on the medical image(s), for example an annotation task.

The downstream task may require certain resources. For example, the downstream task (e.g., annotation) may require certain computing resources. Depending on the CCI, different computing resources (hardware-based and/or software-based) may be allocated for the downstream task, e.g., with respect to: resolution of an image visualization device, e.g., monitor, processing capacities (e.g., for image processing), allocation of time for the task,
human resources with a certain skill level, software licenses (needed for the downstream task), bandwidth for data transfer (both uplink and/or downlink), and/or further resources, for example hardware resources.

The downstream task may be configured or controlled by evaluating the CCI. For example, the computing resources, mentioned above, such as processing capacities and/or the resolution of the e.g. monitor may be configured or the appropriate computing resources are selected based on the CCI. Thus, the appropriate computing resources may be determined and allocated as a preparatory measure to enhance the whole procedure. Alternatively or in addition, it is possible to configure the computing resources on a system, that has been identified to be available. Thus, e.g., variable computing resources may be adapted according to the CCI. For example, software licenses and bandwidth and/or other resources may be switched on or off or may be added or reduced according to actual need, that is determined according to the determined CCI.

Further, the required execution time for the downstream task, for example the annotation time, may be estimated and/or the skill level of the e.g. annotation task may be estimated. Thus, embodiments serves to improve quality by allocating the appropriate resources (time, skill level of the annotator, and/or computing resources for annotation) for the downstream tasks, for example annotation task.

A downstream task may include or be an image-related medical processing task, such as a reporting task or a task required for reporting, such as an annotation task, an image processing task, a selection task, and/or another software-based task that is to be executed on the image.

In another aspect, embodiments provide a computing device for executing the training method as mentioned above.

In another aspect, embodiments provide a CCI-calculator device, that is configured to calculate a case complexity index, CCI. The CCI-calculator device may be configured to execute a method for calculating the case complexity index as described above. The CCI-calculator device may include: an input interface for receiving an input dataset, an output interface which is configured to provide the calculated case complexity index, CCI and a memory for storing a trained neural network, that has been trained with a method as described above with any alternative implementation.

In another aspect, embodiments provide a computer system for use in medical technology for applying a case complexity index, CCI, for an image-related medical processing task, including: an input interface for receiving an input dataset, for example at least one medical image and for example a related report; a calculator interface to a CCI-calculator device as described above for calculating the case complexity index, CCI; and a control interface for controlling the image-related medical processing task based on the calculated CCI.

The CCI-calculator device may be part of the system. For example, the CCI-calculator device may be part of or may be implemented on the computing device (entity) which is configured to calculate the CCI. In addition or alternatively, the CCI-calculator device may be part of the system but implemented on another computing entity which is (only) connected via data connection (wired or wireless, for example via an internet-based protocol, or LAN, WLAN, and/or radio transmission). In this case, the system includes a calculator interface for connecting to the CCI calculator device. It is also possible that the CCI calculator device is connected to the system with an internal connection.

The CCI-calculator device may be part of the computing entity on which the downstream task is to be executed. Alternatively, the CCI-calculator device may be implemented on the image acquisition device or a related computing device.

The system may be implemented in a distributed manner, so that part thereof are implemented on a first computing entity and other parts thereof may be implemented on another computing entity. For example, the trained NN of the CCI-calculator device may be implemented locally or remotely.

In another aspect, embodiments provide a computer program product including program elements that cause a computing device to perform the steps of the method for training a NN or the steps of the method for calculating the CCI as described above when the program elements are loaded into a memory of the computing device.

In another aspect, embodiments provide a computer-readable medium having stored thereon program elements that may be read and executed by a computing device to perform steps of the method for training a NN or the steps of the method for calculating the CCI as described above when the program elements are executed by the computing device.

### BRIEF DESCRIPTION OF THE FIGURES

FIG. 1 depicts a schematic flow chart of a training method according to an embodiment.

FIG. 2 depicts a schematic flow chart of a method for calculating the case complexity index, CCI according to an embodiment.

FIG. 3 depicts a block diagram, schematically representing a case complexity index calculator device according to an embodiment.

FIG. 4 depicts a system for applying the CCI according to an embodiment.

FIG. 5 depicts a training phase and example embodiments of a training method.

FIG. 6 depicts an inference phase and example embodiments of a method for calculating the CCI.

FIG. 7 depicts an implementation of a system for using the determined CCI according to an embodiment.

FIG. 8 depicts a table in matrix structure for calculated CCIs for different input data according to an embodiment.

### DETAILED DESCRIPTION

Embodiments provide an objective metric for calculating a complexity index for a medical image and methods and systems to provide such a complexity index, in the form of a case complexity index, CCI.

Medical images may require a set of subsequent tasks to be performed after imaging, that may also be denoted as downstream tasks, such as annotating, selecting for example for report generation, evaluating, and others tasks. Other potential downstream tasks may include: image reconstruction tasks (e.g. in CT: different kernels, slice thickness, projections), image analysis for all compartments in the images including e.g. detection, characterization, measurement of findings, comparison of current and prior studies, report generation, archiving of imaging data and results and/or quality assurance.

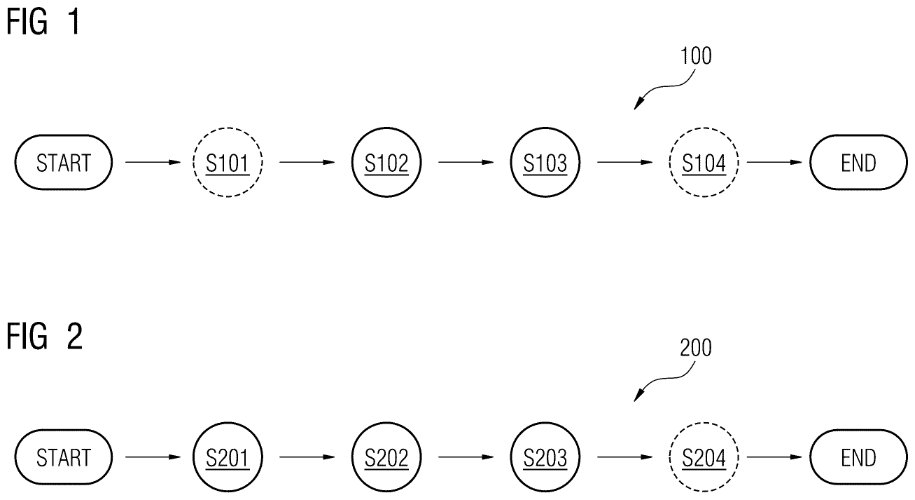

FIG. 1 depicts a computer-implemented method 100 for training a neural network for determining a case complexity index, CCI.

After the start of the method, in a step (shown in dotted lines) S101 the training data may be preprocessed before being used for training, for example the training data may be calibrated.

In step S102 training data (in raw or preprocessed form) are received. Training data may include of a medical image I out of a set of medical images (for example a study for a patient). In an embodiment, the training data may in addition include a report, that refers or relates to the image. The training data may further include a CCI for the medical image. The CCI used in training may be determined algorithmically and is associated to the image I.

In step S103 the NN is trained for providing a trained NN, that is configured for determining the CCI for a medical image I and for example for a related report R by adjusting weights and biases of the NN such that a loss function is minimized.

In an optional step S104 the trained neural network is provided and may be used in inference for new (unseen) images I to determine the CCI for the image I.

After this, the method may end or may be trained on new data.

FIG. 2 depicts a method 200 for using the trained model, for example the trained NN, in an inference phase. After start of the method, in step S201 an input dataset is received. The input dataset includes at least one medical image I and may for example include a medical report for the image. In other embodiments, the NN may be trained on additional data, for example clinical data, operational data, and/or guideline data. Thus, these data types may serve as input dataset as well in inference phase. In step S202 the trained NN is used for calculating the CCI for the received image I. In step S203 the calculated CCI is provided as output. This may be done on a human machine user interface, HMI and/or on other output devices. In addition or alternatively, the calculated CCI may be transferred to other computing entities for further processing. The calculated CCI may for example be used in step S204 (shown in dotted lines, because not mandatory) to configure and/or control downstream processes, for example software-based or software-supported processes on the image I, for example an annotation task. After this, the method may be reiterated or may end. Re-training is also possible.

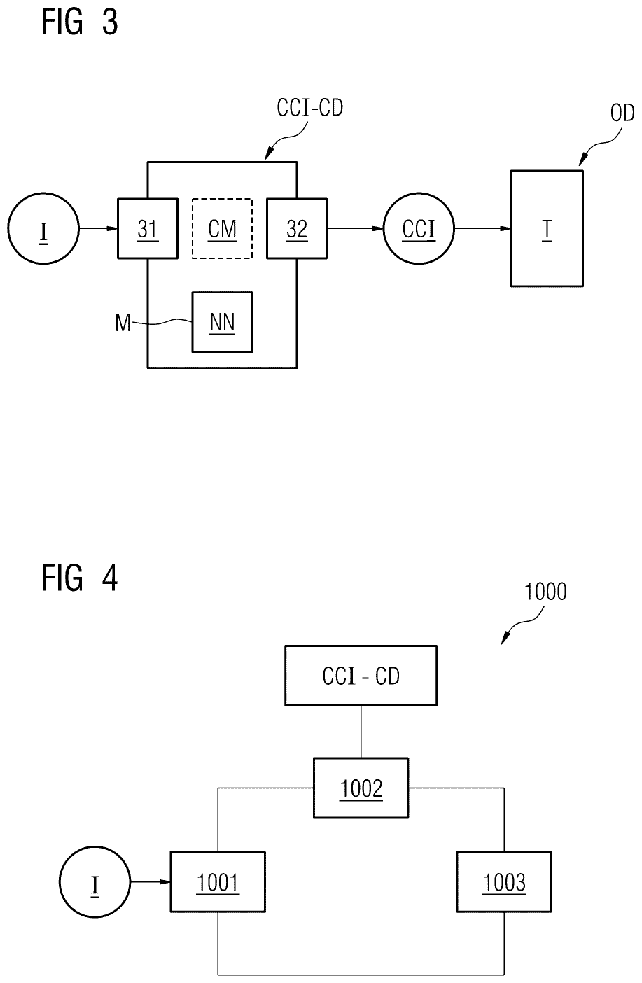

FIG. 3 depicts a schematic drawing of a CCI-calculator device CCI-CD, that is configured to calculate a case complexity index, CCI. The CCI-calculator device CCI-CD may be configured to execute the method 200 for calculating a CCI. The CCI-calculator device CCI-CD may be implemented as hardware and/or software. The CCI-calculator device CCI-CD may be implemented on a CPU or GPU. The CCI-calculator device CCI-CD may include an input interface 31 for receiving an input dataset; an output interface 32 that is configured to provide the calculated case complexity index, CCI; and a memory M for storing a trained neural network NN, that has been trained with a training method as described above. The CCI-calculator device CCI-CD may be interconnected to other devices OD, that are configured to execute the downstream task T, for example an annotation task. The calculated case complexity index, CCI may be used to open or closed loop control or configure the downstream task T. The CCI-calculator device CCI-CD may include a calibration module CM on an optional basis (dotted lines).

FIG. 4 depicts an example computer system 1000 system for use in medical technology for applying a case complexity index, CCI, for an image-related medical processing task, including an input interface 1001 for receiving an input dataset, for example including at least one medical image I and for example a related report R; a calculator interface 1002 to a CCI-calculator device CCI-CD as mentioned above for calculating the case complexity index, CCI; and a control interface 1003 for controlling the image-related medical processing task (downstream task T) based on the calculated CCI.

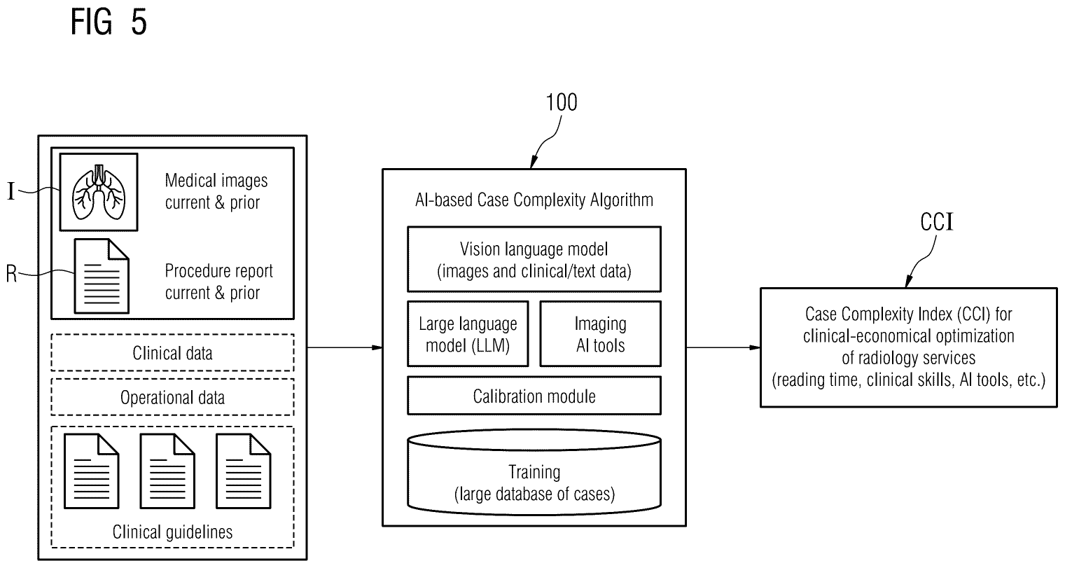

FIG. 5 depicts an example embodiment of the training method 100. Input to the training in form of training data are at least images. Alternatively training data may further include a report R for the image. Thus, a pair of training data may be provided, for example pairs of images and (e.g. procedure) reports. Alternatively, further training data may be provided and processed, for example clinical data, operational data, and/or clinical guidelines.

Clinical data may include e.g. patient demographics, known diagnoses, co-morbidities, therapy related information and lab values. Operational data in the retrospective training phase may include experience/skills of reading physicians or medical annotators, reading/annotating times as well as referral sources and external data, such as Relative Value Units, RVU, for an exam.

Relative Value Units (RVU) are assigned to specific procedures to determine physician reimbursement and do reflect factors such as time, intensity, and cost of care. RVUs may also be used to determine physician productivity. However, RVU are determined by a committee approach with an update cycle of several years. Comprehensive and continuous analysis of all relevant input data with the CCI may enable more granular and accurate results to drive clinical-economical optimization, e.g. assignment of cases to available readers.

Joint training on imaging and text data may be done with vision-language processing approaches. For more details it is referred to https://www.frontiersin.org/articles/10.3389/frai.2019.00028/full.

The presence of complementary and contradicting information between image data and reports may be utilized to determine case complexity across large numbers of cases even without knowing the exact ground truth in each case. Procedure reports may also contain inaccuracies and errors.

The reporting style (structure, level of detail, terminology) is expected to vary across physicians and sites. Different words may have the same meaning while small differences in wording may imply very different clinical meaning. Use of “uncertainty language” in imaging procedure reports is very common, e.g. “lung nodule may be ruled out.” Therefore, reporting style and uncertainty language in retrospective procedure reports may be processed by large language models (LLM) and used in the training of the CCI algorithm, i.e. the training method.

Comparison with prior images is standard of care and may be trained using retrospective cases with current and prior exams. This includes the impact of priors on reading time when compared to cases without prior exams.

Training may include approaches to distinguish cases with common findings from cases with rare findings in the long-tail distribution without the explicit need to detect or characterize the specific finding or disease.

Imaging AI tools may include algorithms to detect cases with specific findings as well as cases with non-actionable disease.

The Calibration Module CM may be implemented as solving an optimization problem. The Calibration Module CM may be configured taking into account site specific data.

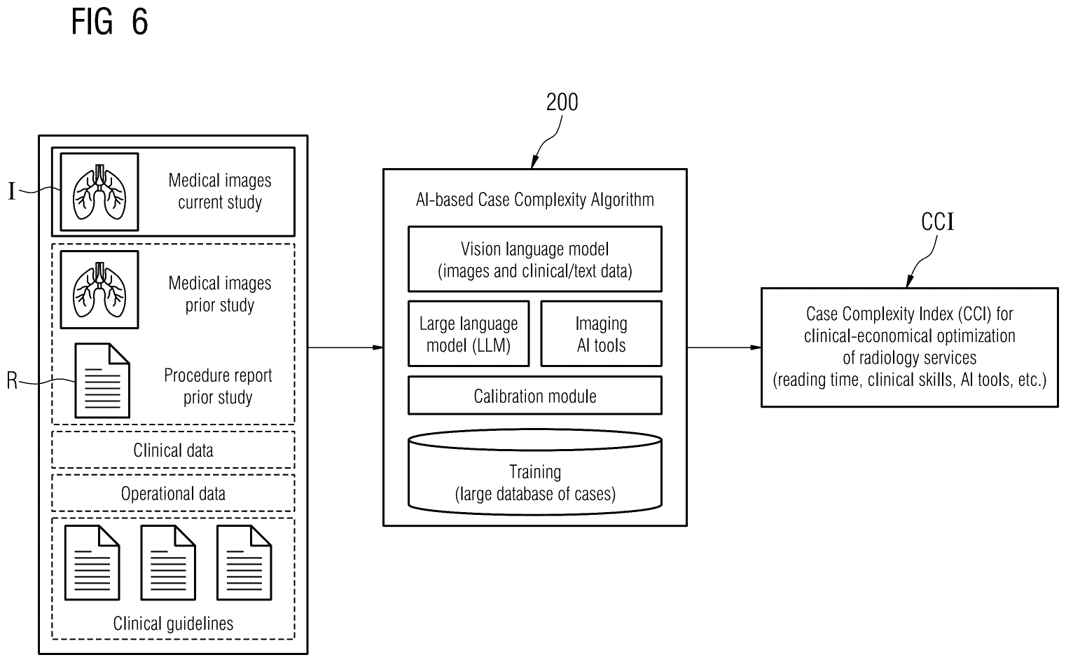

FIG. 6 depicts the method 200 for calculating a CCI in an inference or deployment phase. The method 200 or algorithm processes medical images from the current study that need to be read (required input) as well as images and reports from the prior study or studies, and for example clinical data, operational data, and/or clinical guidelines. The input dataset include medical images I and a related medical report R. Thus, the input dataset may include of a multimodal dataset, consisting of image data and text data. In this scenario, the training method 100 needs to be trained with images I and reports R as well.

If the input dataset only includes images I in the inference method 200, the training method 100 correspondingly needs to be trained with images.

Prior studies may refer to the same or a related procedure type. For example, a current chest radiograph may have a chest CT as the prior exam.

Clinical data in the prospective deployment phase may include the reason for the exam, known diagnoses, and related information such as lab values.

Operational data in the prospective deployment phase may include e.g. the referral source for the procedure.

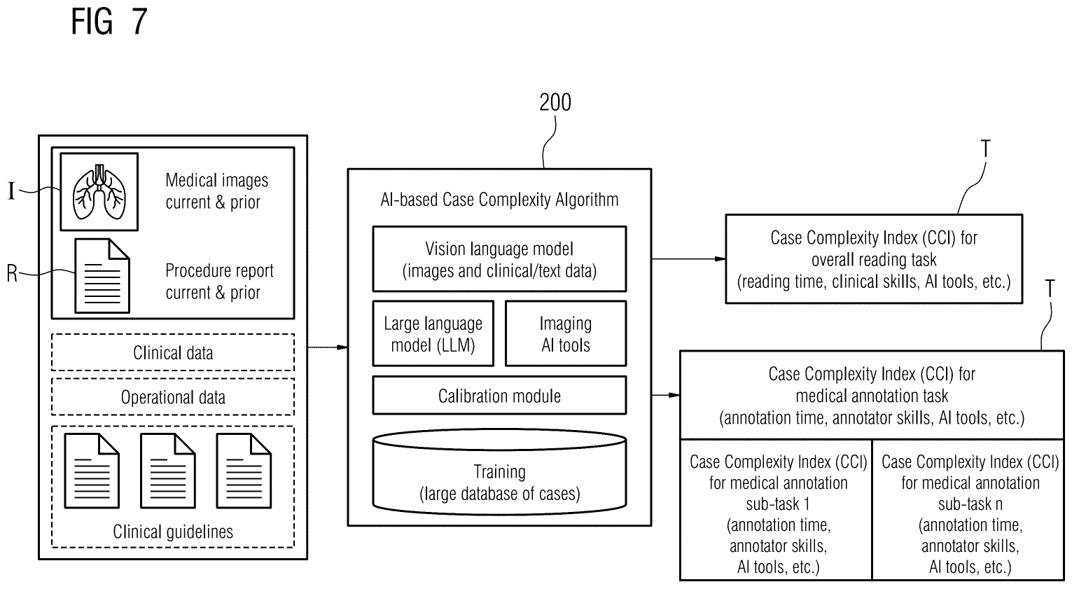

FIG. 7 depicts in an example embodiment, the use of the calculated CCI for the downstream tasks T performed by medical annotators performing image review and/or annotation services towards a radiology report or medical image.

The calculated CCI may be used to match the skills of the annotators (e.g. basic non-physician, advanced non-physician, radiologist, subspecialty radiologist) to specific cases or tasks and to measure productivity while adjusting for case difficulty.

The CCI for the overall annotation task T (e.g. annotating a chest CT with all findings) may be broken down into multiple annotation sub-tasks (e.g. detecting lung nodules, measuring their size, etc.).

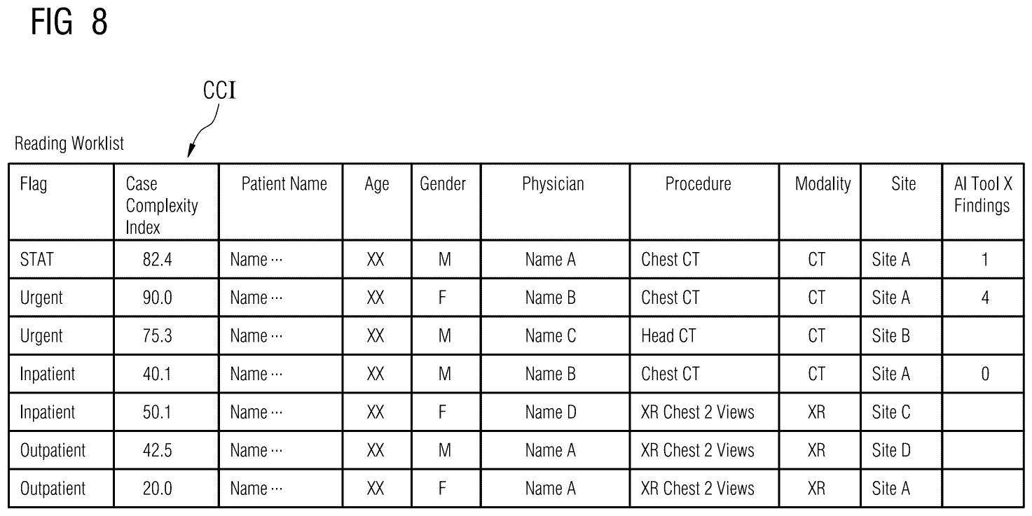

FIG. 8 depicts a representation of a calculated case complexity index, CCI. The CCI may be part of the reading worklist of an image service provider. The CCI may be used across different imaging studies and multiple sites. The CCI may also be used by an imaging service provider serving multiple customers sending cases for reading (e.g. teleradiology service), where customer requirements for reading and reporting may vary substantially. This also includes RaaS offerings using medical annotators to prepare preliminary reports.

In an embodiment, the CCI may be used for specific imaging procedures, e.g. chest CT exams or chest radiographs.

In an embodiment, the CCI provides a sub-index for reading time and reading difficulty.

The AI-based calculation of the case complexity index, CCI may be systematically and proactively used for technical optimization, such as optimized assignment of cases to readers and/or configuration of devices for the execution of the downstream task T and/or worklist prioritization. The proposed CCI has the potential to be highly accurate due to the comprehensive scope of input data. The proposed CCI is also sustainable over time as the training may be updated to reflect technology improvements, new reporting requirements or other learnings from providing imaging services.

The CCI may be used as part of a Radiology as a Service implementation to execute reading tasks as efficiently as possible while ensuring highest possible quality of care. It may also be provided to customers as part of an imaging IT solution.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer implemented method for training a neural network for determining a case complexity index, CCI, the method comprising:
receiving training data, the training data comprising pairs of: a medical image of a set of medical images and a CCI for the medical image; and
training the neural network with the training data to determine a respective CCI for a respective medical image by adjusting weights and biases of the neural network such that a loss function is minimized.

2. The computer implemented method of claim 1, wherein the training data further comprises a related report for the medical image.

3. The computer implemented method of claim 1, wherein the training data further comprises at least one of: clinical data for a respective patient, what the medical image refers to, operational data, and/or guideline data.

4. The computer implemented method of claim 1, wherein the set of medical images comprises current images, prior images, or current images and prior images of a same procedure, a same patient, or the same procedure for the same patient.

5. The computer implemented method of claim 1, further comprising:
calibrating the received training data using a calibration module with respect to different images, reports, or different images and reports.

6. The computer implemented method of claim 1, wherein the neural network comprises at least one of a vision language model, a large language model, or an image processing model.

7. The computer implemented method of claim 1, further comprising:
calculating a case complexity index for an input dataset comprising at least one medical image and a related report for the at least one medical image using the trained neural network.

8. The computer implemented method of claim 7, wherein the calculated CCI is used for configuring a software-based downstream task on the medical image.

9. The computer implemented method of claim 8, wherein the software-based downstream task comprises an image annotation task, wherein the calculated CCI is used with respect to estimated reading time, and/or clinician skill level.

10. A device configured to calculate a case complexity index, CCI, the device comprising:
an input interface configured for receiving an input dataset;
an output interface configured to provide a calculated case complexity index;
a memory for storing a trained neural network configured to calculate a case complexity index for an input dataset comprising at least one medical image and a related report for the at least one medical image using the trained neural network.

11. A system for use in medical technology for applying a case complexity index, CCI, for an image-related medical processing task, the system comprising:
an input interface configured for receiving an input dataset comprising at least one medical image and a related report;
a calculator interface for a CCI-calculator device configured for calculating the case complexity index using a trained neural network when provided the input dataset; and
a control interface configured for controlling the image-related medical processing task based on the calculated CCI.

12. The system of claim 11, wherein the trained neural network is trained with training data to determine a respective CCI for a respective medical image by adjusting weights and biases of the trained neural network such that a loss function is minimized, the training data comprising sets of: a medical image of a set of medical images, a related report for the medical image, and a CCI for the medical image.

13. The system of claim 12, wherein the training data further comprises at least one of: clinical data for a respective patient, what the medical image refers to, operational data, and/or guideline data.

14. The system of claim 12, wherein the set of medical images comprises current images, prior images, or current images and prior images of a same procedure, a same patient, or the same procedure for the same patient.

15. The system of claim 11, wherein the trained neural network comprises at least one of a vision language model, a large language model, or an image processing model.

16. The system of claim 11, wherein the calculated CCI is used for configuring a software-based downstream task by the control interface.

17. The system of claim 16, wherein the software-based downstream task comprises an image annotation task, wherein the calculated CCI is used with respect to estimated reading time, and/or clinician skill level.
